(19) United States
(12) Patent Application Publication
(10) Pub. No.: US 2022/0245227 A1
(43) Pub. Date: Aug. 4, 2022

(54) SYSTEMS AND METHODS FOR USER VERIFICATION BASED ON ACTIGRAPHY DATA

(71) Applicant: Novartis AG, Basel (CH)

(72) Inventors: Jonas DORN, Basel (CH); Vittorio Paolo ILLIANO, Basel (CH)

(21) Appl. No.: 17/619,227

(22) PCT Filed: Jun. 16, 2020
(86) PCT No.: PCT/EP2020/066564

§ 371 Date: Dec. 14, 2021

(30) Foreign Application Priority Data
Jun. 21, 2019 (EP) 19181740.2

(51) Int. Cl. G06F 21/32; G06K 9/62; G06F 21/34

(52) U.S. Cl. CPC G06F 21/32; G06K 9/6265; G06F 21/34; G06K 9/6277

(57) ABSTRACT

The invention provides systems and methods for providing a user-specific activity model based on actigraphy data and for user verification based on a user-specific activity model based on actigraphy data.

The invention is directed to systems and methods for providing a user-specific activity model based on actigraphy data and for user verification based on a user-specific activity model based on actigraphy data.

Actigraphy data is data related to a user's activity and can be obtained, for example, by means of a wearable device worn by the user. Actigraphy data may comprise accelerometer data detected by accelerometer sensors.

Actigraphy data can be used to detect changes in the physical condition, for example health status, of the person wearing the wearable device. Thus, a remote monitoring and/or analysis of the physical condition and, in particular, changes thereof over time is possible.

In view of this, the user need not be in a controlled or supervised environment in order to monitor the physical condition. For example, the user can wear the device at home.

One application of actigraphy data monitoring is use in clinical trials in the pharma industry, where sensors are used to collect objective measurements on disease symptoms and patient behavior. Clinical trials can be improved by making them remote, i.e., allowing the participants to be unsupervised as described above.

While this is advantageous for many reasons, it also brings about the challenge that due to the lack of supervision, it is desirable to provide a way of verifying that the data obtained by the device actually belong to the user who claims to be wearing it, that is, whether only the expected user wears the device while the actigraphy data is being obtained. Without such a verification, an impostor might be wearing the device and, thus, distort the results of an evaluation of the data and lead to wrong conclusions regarding the expected user's physical condition.

The problem of identifying impostors has been met with different user verification methods. These methods generally include using additional, non-actigraphy data, based on which a user verification is carried out. Such data may comprise biometric data. For example, the additional data may comprise iris scans or fingerprint scans.

However, obtaining additional data requires additional sensing techniques and is also not necessarily as reliable as is required for certain applications, in particular, as some of the methods would enable the expected user to perform the verification and then hand the device over to an impostor.

Therefore, a problem underlying the present invention is to provide systems and methods that allow for identifying a user based on data that is also suitable for monitoring the physical condition of the user and performing user verification based thereon.

The invention provides a computer-implemented method for providing a user-specific activity model, in particular for user verification, the method comprising obtaining actigraphy data from a plurality of users and determining the user-specific activity model of a first user of the plurality of users based on the actigraphy data obtained from the first user and a reference actigraphy data set comprising actigraphy data of the remaining users of the plurality of users. The actigraphy data may be obtained by means of one or more wearable devices.

The determining of the user-specific activity model may comprise processing the data of the first user and the data of the remaining users separately and then merging the processed data to obtain the activity model. Alternatively the data of the plurality of users may first be merged and then processed together to obtain the activity model.

The invention further provides a computer-implemented method for user verification comprising obtaining actigraphy data by means of a wearable device, verifying, based on a user-specific activity model of a first user, which is based on actigraphy data of the first user obtained during a first period of time, whether actigraphy data obtained during a second period of time subsequent to the first period of time belongs to the first user, and if it is determined that any of the actigraphy data obtained during the second period of time does not belong to the first user, marking the data that does not belong to the first user as impostor data and/or raising an alarm indicating that impostor data was detected. The obtaining of actigraphy data during the first period of time may be but is not necessarily part of said method. It may be but is not necessarily performed by the same wearable device.

In particular, the method for user verification may comprise the method for providing a user-specific activity model and use the activity model obtained accordingly for the step of verifying whether actigraphy data obtained during the second period of time belongs to the first user.

The user-specific activity model may be based on activities detected in the actigraphy data, in particular, on activities that are determined as being characteristic of the user. The user-specific activity model may include information identifying different activities, and in particular may reflect how said activities are performed and/or in which pattern, e.g. at which frequency, they are performed. The identification of activities may be on an abstract level that does not require attributing the activities to specific user actions. For example, it is not necessary to identify that a certain activity corresponds to the actual act of writing or brushing teeth. The activity model may be seen as a fingerprint of activities. The activity model captures the characteristic activities of an expected user that differentiate the expected user from other persons. The user-specific activity model may, for example, include typical and/or atypical movement patterns.

The first period may be seen as a control or reference period. During the first period of time, the first (expected) user may optionally be monitored by additional devices and/or personnel so as to ensure that the expected user is actually wearing the wearable device.

The first period of time may be a time during which only the first user wears the wearable device. This may be determined and/or ensured, for example, when the first user is fully monitored during the first period of time.

Alternatively, the first period of time may comprise times when the first user wears the wearable device and may also comprise times when another user wears the wearable device. For example, this may be the case when the first user temporarily hands over the wearable device to another user.

The method may comprise determining a plurality of preliminary activity models from the actigraphy data obtained during the first time period and employing the plurality of preliminary activity models to obtain the user-specific activity model, i.e., the activity model to be used in the above described step of verifying whether actigraphy data obtained during the second period of time belongs to the first user. This is particularly useful when the first user is not fully monitored during the first period, such that it cannot be confirmed with certainty that the first period of time is a time during which only the first user wears the wearable device.

The plurality of preliminary activity models may be used to obtain the user-specific activity model by generating a consensus activity model, for example by aligning the plurality of preliminary activity models and using only the typical and/or atypical activities that are common to all preliminary activity models to create the consensus activity model.

Alternatively, the plurality of preliminary activity models may be employed to remove a portion of the actigraphy data obtained during the first period of time to obtain a reduced set of actigraphy data.

The reduced set of actigraphy data may then be used for obtaining the user-specific activity model. That is, rather than using all the data obtained during the first period of time, only a part thereof is used for obtaining the user-specific activity model.

The method may comprise creating the reduced set of actigraphy data by removing actigraphy data identified as likely impostor data from the actigraphy data obtained during the first period of time. Actigraphy data may be identified as likely impostor data by means of the plurality of activity models.

As an example, the method may comprise dividing the first period of time into two or more, particularly non-overlapping, sub-periods. In this case, the method may comprise providing a preliminary activity model for each of at least two of the sub-periods of the first period using only actigraphy data obtained during the respective sub-period. Thus, the plurality of preliminary activity models may be provided.

For example, the method may comprise providing a first preliminary activity model based on data of a first sub-period of the first period and providing a second preliminary activity model based on data of a second sub-period of the first period. The preliminary activity models may each be obtained using any of the methods described herein for the obtaining an activity model.

The method may comprise using the first preliminary activity model to identify actigraphy data that is likely to be impostor data among the actigraphy data obtained during the second sub-period and using the second preliminary activity model to identify actigraphy data that is likely to be impostor data among the actigraphy data obtained during the first sub-period. The identifying may be performed by means of any of the methods described herein for verifying whether actigraphy data belongs to the first user.

Each of the sub-periods may be a random set of times, for example a random set of days, within the first period, optionally with the limitation that the sub-periods are non-overlapping as mentioned above. Each of the sub-periods may optionally be a contiguous time period, for example a plurality of consecutive days, or a non-contiguous period, for example a plurality of non-consecutive days.

Although the above examples relate to two sub-periods, the method for obtaining a reduced set of actigraphy data can also be performed using more than two sub-periods and more than two preliminary activity models.

The use of a plurality of preliminary activity models for determining the user-specific activity model may increase robustness to impostors during reference data collection.

The second period may be performed with less than or no monitoring by additional devices and/or personnel. For example, this may be the period of a remote medical trial. Of course it is possible to have some intermittent additional monitoring in place during this period, but no continuous additional monitoring needs to be performed.

The wearable device may be any device that comprises sensors, which, at least when worn by a user, provide sensor data including actigraphy data. For example, the device may be intended for being worn around the wrist, neck, ankle or attached to any other body part. Wrist-worn devices are particularly suitable for capturing characteristic activities. Actigraphy data may comprise data that reflects the activities performed by the user while wearing the wearable device. In particular, as indicated above, actigraphy data may comprise accelerometer data detected by an accelerometer sensor. In particular, it may comprise acceleration values and values indicating the time at which the respective acceleration values were obtained. That is, the wearable device may comprise an accelerometer sensor configured to provide actigraphy data including accelerometer data.

An activity as used herein may refer to single movements of one or more body parts of the user, for example lifting an arm, as well as a superposition and/or concatenation of movements, for example walking, eating, writing, typing, or brushing one's teeth.

The method may comprise collecting actigraphy data continuously while the wearable device is worn by a user and switched on. In particular, the actigraphy data may be collected continuously during the entire first and/or second period of time. As an example, the continuous measurement may comprise that the actigraphy data is collected at a sampling rate in the range of 10⁻² Hz and 10³ Hz, 10⁻² Hz and 300 Hz in particular 10⁻¹ Hz to 10² Hz, in particular 1 Hz and 80 Hz, in particular 10 Hz and 60 Hz, in particular 20 Hz and 40 Hz, in particular 25 to 35 Hz, in particular 30 Hz. Examples for suitable sampling rates include 0.017, 0.2, 1, 20, 25, 30, 32, 60, 80, 100, and 256 Hz.

Anybody wearing the wearable device while it is switched on is deemed to be a user of the device. The first user is referred to as the expected user. This may, for example, be the user to be monitored during the second period, e.g., during the clinical trial. Any user who is not the first user will in the following be referred to an impostor.

The method uses the actigraphy data provided by the wearable device to dynamically determine whether the actigraphy data belongs to the first user, i.e., the expected user, or an impostor. This determination can be considered as a user verification.

The method does not require repeatedly calculating a new activity model based on data obtained during the second period of time and comparing it to the user-specific activity model based on data obtained during the first period of time. It rather comprises that it is judged from actigraphy data measured during the second period of time whether this would rather fit the activity model of the expected user during a first period of time or an impostor.

The advantage of the claimed authentication method is that it reliably allows for impostor detection without providing any additional sensors for verification than the sensors that can be used for monitoring the physical condition of the current user, e.g., the health status. Moreover, with respect to some known methods, e.g. iris or finger scan, it is also more secure, as the expected user cannot deliberately authenticate an impostor.

The method may comprise triggering an alert. This would allow confirmation by other means than the actigraphy data whether the deviations exceeded the threshold due to rapid changes of the physical condition, which may imply danger to the expected user, or due to the wearable device having been transferred from the expected user to an impostor. Generally, the expected deviations due to changes in the physical condition, for example when a drug is showing the intended effect or the user exhibits any side effects, are expected to be gradual. Moreover, some activities will remain similar in spite of changes to the physical conditions. Therefore, the method allows for reliably distinguishing whether a change is due to an impostor or due to changes in the user's physical condition.

The method, particularly a creation of the activity model and/or the verification, may be carried out entirely by the wearable device or it may at least partially, particularly completely, be evaluated on one or more remote devices. The verifying may be performed during the second period, particularly continuously, and/or the data may be subject to the verification at some later time. In that case, in particular, all the data collected during the second time may be evaluated collectively.

The wearable device may provide actigraphy data to the remote device based on a push and/or pull scheme. That is, the wearable device may, continuously or discontinuously provide actigraphy data to the remote device of its own motion. Alternatively or in addition, the wearable device may provide actigraphy data to the remote device in response to a request received from the external device, some other device, or as prompted by a person.

Briefly summarized, the method may include grouping, also referred to as clustering, actigraphy data into activity clusters, possibly after some filtering and/or structuring thereof, using the actigraphy data from the first period (reference period), building a dataset from the clustered data, extracting the characteristics of the expected user using the activity clusters, and building a probabilistic model that captures the difference between the expected user and a generic impostor. Actigraphy data obtained during the second period may be used to update, based on the probabilistic model, the confidence in the user's identity, e.g., based on the observation of each activity. These steps are presented in more detail below.

The methods may comprise adding actigraphy data obtained by the wearable device to a candidate set of actigraphy data and structuring and/or filtering the actigraphy data of the candidate set to obtain a data set to be used for creating the activity model and/or for the verifying step.

The structuring may comprise dividing the actigraphy data of the candidate set into consecutive finite time windows, particularly adjacent, non-overlapping windows.

The time windows may have a fixed size W. The advantage of such a fixed size is that no additional information like start and end of an activity are required to define the time windows. In this case, it is advantageous that, in the step of clustering of time series, which will be described in more detail below, a cross-correlation method between two time series comprises zero padding in the regions where they do not overlap.

Alternatively, the windows may have a variable size. The variable size may be determined using determination of start and end of an activity, for example by means of activity segmentation.

The size of the time window W may be optimized to minimize trimming of a unique activity and blending different activities together. The window size may be optimized as a function of the sampling rate. For a given sampling rate, there is a trade-off between the number of samples in the window and the window size. That is, the shorter the window, the fewer samples are in the window. For a given sampling rate and number of samples in the window, the window size may be equal to the number of samples in the window divided by the sampling rate.

The number of samples in the window may be between 1 and 1800, in particular, between 3 and 1650, in particular, between 5 and 1500, in particular between 10 and 1350, in particular between 20 and 1200, in particular between 30 and 1050. Examples for a suitable number of samples in a window include 1, 3, 5, 10, 20, 30, 900, 1050, 1200, 1350, 1500, 1650 and 1800 samples. For example, when a sampling rate is in the order of one sample per minute, the number of samples in the window may be in the order of 10⁰ to 10¹. As another example, when a sampling rate is in the order of 10¹ samples per second, e.g. 30 Hz or 50 Hz, the number of samples in the time window may be in the order of 10² to 10³. As an example, the sampling rate may be 30 Hz and the number of samples in the window may be 900, resulting in a window size of 30 seconds.

The filtering may comprise a step of removing data that is categorized as invalid inactivity data from the candidate set, in particular all data that is categorized as invalid inactivity data.

The categorization of data as invalid inactivity data may comprise determining a standard deviation of actigraphy data, in particular the magnitude of the measured data, e.g. accelerometer data, in a given time interval and determining whether the standard deviation exceeds an activity threshold Tₐ.

The given time interval may correspond to the time window. Alternatively, the time window may be divided into M, particularly contiguous, a plurality of sub-windows and the given time interval may correspond to one of the sub-windows.

The data of a given time interval may unconditionally be categorized as invalid inactivity data when it is determined that the standard deviation does not exceed the activity threshold Tₐ.

Alternatively, it may be determined, for a group of contiguous time intervals, that less than a predefined number of said contiguous time intervals have a standard deviation exceeding the activity threshold, and, in response, all time intervals of said group may be categorized as invalid inactivity data.

When it is determined that equal to or more than the predefined number of said contiguous time intervals have a standard deviation exceeding the activity threshold, the data of each time interval of the group having a standard deviation exceeding the activity threshold may be categorized as valid data and the data of all the remaining time intervals of the group may be categorized as invalid data.

The predefined number may for example be 60%, in particular 50%, in particular 40% of the number of time intervals in the group.

As an example, all the sub-windows of one time window may constitute the group of contiguous time intervals. Thus, the data of an entire window may be categorized as invalid data when less than the predefined number of all of its sub-windows have a standard deviation exceeding the activity threshold. That is, all the data of a time window may be discarded if it includes valid and invalid data.

This allows for avoiding categorization errors, e.g. stemming from non-stationary signals.

Alternatively or in addition to the analysis of the standard deviation, invalid inactivity data may also be identified by means of a non-wear detection algorithm. For example, the method described in Vincent T Van Hees et al. “Separating movement and gravity components in an acceleration signal and implications for the assessment of human daily physical activity” (PLOS ONE 8.4 (2013), e61691) may be used.

The filtering may comprise characterizing a sub-set of data within the candidate set as good data only when the proportion of data removed from the sub-set exceeds a threshold Tar and/or only when the sub-set is part of a group of similar sub-sets occurring repeatedly in a specific pattern, and adding only the good data to a final data set, wherein the final data set is used, in particular, for creating the activity model.

In other words, one or more criteria are applied to the data so as to determine good data to be used, for example for creating the profile. The criteria of proportion of removed data and repeated occurrence in a specific pattern are referred to as primary criteria in the following.

The filtering allows for avoiding distortions and improves the meaningfulness of the data set used.

In particular, the entire first and/or second period may each be divided into equal sections or time spans, e.g. an hour, and the sub-set of data is defined as the data of one of the sections. Each section may be characterized as a good section or a bad section. The characterization as a good section may be based on the proportion of data previously removed from the candidate set, for example the invalid inactivity data, and remaining data within said section. The characterization step may include comparing the proportion of a section to a threshold Tₐᵣ and characterizing the section as a good section only if the proportion exceeds the threshold. When the proportion does not exceed the threshold, the section is characterized as a bad section. The remaining data (i.e., not previously removed from the candidate set) in a good section is characterized as good data and the remaining data in a bad section is characterized as bad data. Such filtering particularly reduces distortion due to excessive removal of data.

As seen above, in addition or alternatively, a sub-set of data is characterized as good data only when the sub-set is part of a group of similar sub-sets occurring repeatedly in a specific pattern. The characterization as a good section may then be based on whether sections with a similar activity distribution occur repeatedly in a specific pattern, particularly at a specific frequency, for example, every 24 hours.

Such filtering allows for choosing particularly characteristic sections or sub-sets of data, as they indicate habits of the user wearing the device.

When a characterization is made based on the proportion of data removed from the sub-set and based on whether the sub-set is part of a group of similar sub-sets occurring repeatedly in a specific pattern, this means that the sub-set is only characterized as a good data when the criteria for both are met.

In particular, the characterization based on the proportion of data removed from the sub-set may be performed first for a plurality of sub-sets and sub-sets with data characterized as good data may be identified. Thus, a plurality of pre-selected sub-sets is determined. Then it is determined whether some of the pre-selected sub-sets are part of a group of similar sub-sets occurring repeatedly in a specific pattern. Data of such sub-sets is characterized as good data.

The methods may comprise a step of selecting data for the reference actigraphy data set among the actigraphy data of the remaining users. That is, for creating the fingerprint, a mixture of data from the expected user and other users may be used as input. As an example, for the expected user Nₑᵤ data samples may be selected from the first period of time. In order to create a reference profile, for each of a number Mᵢᵤ, where preferably Mᵢᵤ>1, of other users the same number of samples from a respective period may be obtained and Nₑᵤ/Mᵢᵤ samples may be drawn at random with uniform or non-uniform probability from these samples.

The method may comprise processing actigraphy data, in particular actigraphy data from the final data set, so as to group activities together to form clusters by means of a three-dimensional time series clustering method, for example based a k-partition clustering method, for example the k-shape method or the k-means method, to provide activity clusters.

In other words, activities are detected in the actigraphy data. The activity model is based on some of the activities detected in the actigraphy data, in particular, on activities that are determined as being characteristic of the user.

Generally, activity recognition is a complex problem, particularly, when the activities' semantics have to be understood. The problem is simplified by inferring whether pieces of data pertain to the same activity using clustering.

Actigraphy data can be divided into a plurality of time series. The time series is a data set including measured actigraphy, e.g., acceleration, values and their respective time. Time series clustering comprises determining a similarity between two time series based on a similarity metric. When the similarity is above a clustering threshold, it is determined that the time series are part of the same cluster.

One time series clustering method known in the art is, for example, the k-shape method (see, for example, John Paparrizos and Luis Gravano. “k-shape: Efficient and accurate clustering of time series”. In: Proceedings of the 2015 ACM SIGMOD International Conference on Management of Data. ACM. 2015, pp. 1855-1870). The similarity metric in this case is the maximum Pearson Correlation. However, actigraphy data generally comprises three-dimensional data, for example acceleration data, and the known methods are not suitable for three-dimensional data. Reducing three-dimensional data to one-dimensional data, e.g. acceleration magnitude, results in loss of information like orientation-related information, e.g., regarding direction of acceleration. Clustering time-series separately on each dimension neglects the correlation between the three axes, which can also be seen as a loss of information.

The three-dimensional time series clustering method may comprise using metrics based on the maximum Pearson Correlation, yet modified to accommodate three-dimensional data. In particular, the similarity metric may be defined as normalized cross-correlation averaged along the three axes and maximized among all possible time-shifts, subject to a maximum shift difference across the three axes.

The claimed feature allows for avoiding the above-described loss of information.

In other words, the similarity metric may be an average between three normalized cross-correlations, one for each axis. Across each dimension, the time shifts are selected to maximize the average cross correlation, subject to a constraint in the maximum distances between the three time shifts. That is, different time shifts are allowed across the three axes.

Different time shifts overcome synchronization errors and allow for more generality in the movements' timing.

The above clustering method leads to activities being performed in a typical way to be grouped into their own cluster. Therefore, each of the clusters, by definition, has a sufficient amount of typicality. Typicality is one indicator as to the quality of an activity as source of evidence in the verification step.

Further indicators of the quality are the frequency at which the activity is performed and the consistency, which describes the stability of the other two criteria and may be taken into account when creating the activity model.

The verifying may comprise inputting actigraphy data obtained during the second period of time into a probabilistic model that defines the probability that the user wearing the device is the first user based on the user-specific activity model and an activity in the actigraphy data, determining whether the probability determined by the probabilistic model is above a first threshold, also referred to as high threshold Tₕ and/or determining whether the probability determined by the probabilistic model is below a second threshold, also referred to as low threshold Tₗ, and determining that the actigraphy data belongs to the first user when the probability determined by the probabilistic model is above the first threshold, and/or determining that the actigraphy data does not belong to the first user when the probability determined by the probabilistic model is below the second threshold, and/or determining that the input data is not sufficient for determining whether the actigraphy data belongs to the first user or an impostor when the first threshold is not exceeded and the second threshold is exceeded.

The probability is also referred to as confidence. The probabilistic model may be configured to calculate the probability of the expected user or confidence given the history of observed clusters until the current time instant t. The probabilistic model may, for example, employ a time-ordered sequence of traversed clusters.

The model may, for example, use a number of time series extracted from the data of the expected user and the same number of time series extracted from data of other users, e.g., as described above in the context of data selection, and calculate the number of occurrences of each cluster for the expected user and other users, respectively.

The methods may comprise building the probabilistic model and fitting the model by counting the number of occurrences for the expected user and other users in each cluster. The method may further comprise performing model refinements, which may include defining a confidence update criterion and defining the criteria for excluding clusters, e.g., when they exhibit inconsistent frequencies.

The probabilistic model may be configured to update the probability when an activity is observed, in particular for each observed activity, and the verifying may comprise repeatedly determining whether the probability determined by the probabilistic model is above a first threshold and/or determining whether the probability determined by the probabilistic model is below a second threshold, in particular, until the probability either exceeds the first threshold Tₕ or does not exceed the second threshold Tₗ.

In addition, or alternatively, the method may comprise a reset of the probability after a predetermined number maxE of updates of the probability.

This allows for disregarding outdated data.

Tₗ, Tₕ, maxE drive the performance of the verification. In practice they may be optimized taking into account the gain in detecting impostors and cost of detecting genuine users as impostors. In many use cases, for example remote medical studies, the aim is to minimize FAR (false alarm rate), while also trying to reduce MTTD (mean time to detection) after the first user hands the device over to an impostor.

The invention also provides a system comprising processing means configured to perform the steps of obtaining actigraphy data of a plurality of users, in particular by means of one or more wearable devices, and determining the user-specific activity model of a first user of the plurality of users based on the actigraphy data of the first user and a reference actigraphy data set comprising actigraphy data of the remaining users of the plurality of users.

The invention also provides a system comprising a wearable device having at least one sensor configured to obtain actigraphy data. The system further comprises processing means configured to perform the following steps: verifying, based on a user-specific activity model of a first user, which is based on actigraphy data of the first user obtained during a first period of time, whether actigraphy data obtained during a second period of time subsequent to the first period of time belongs to the first user, and if it is determined that any of the actigraphy data obtained during the second period of time does not belong to the first user, marking the data that does not belong to the first user as impostor data and/or raising an alarm indicating that impostor data was detected.

This system may be configured to perform the steps of obtaining actigraphy data of the plurality of other users and determining the user-specific activity model of the first user based on the actigraphy data of the first user and the reference actigraphy data set comprising actigraphy data of the remaining users of the plurality of users.

These systems, particularly the processing means, may be configured to carry out any of the above-described method steps, in the above described or any other combination.

The wearable device may comprise the processing means or part of the processing means. The wearable device may comprise a communication interface for communicating, e.g., via a wireless data connection or via a wired connection, with external devices, which optionally may also comprise part of the processing means.

The invention also provides the use of the above system for carrying out any of the above methods.

Features, feature combinations, definitions, and advantages described above in the context of the methods similarly apply in the context of the system.

Further examples of the invention will be described below with reference to the attached figures.

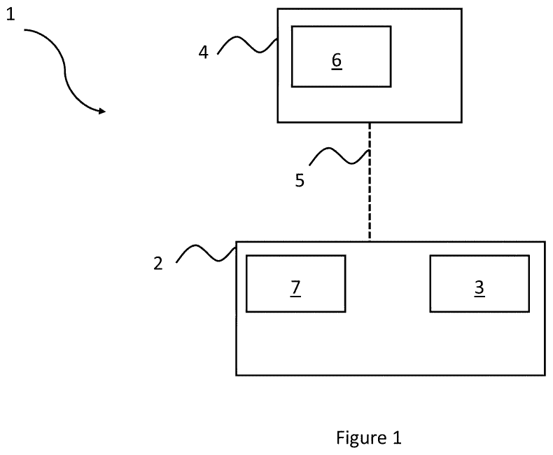

FIG. 1 illustrates a schematic and not-to scale view of a system according to the invention;

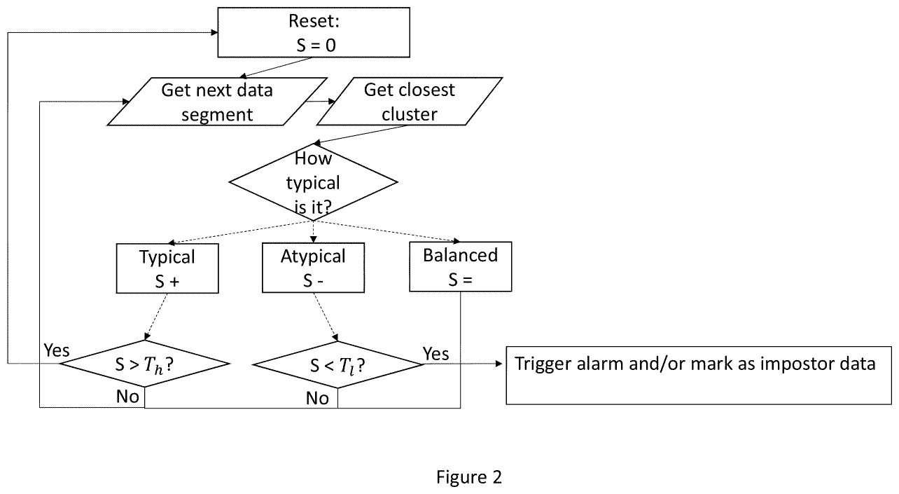

FIG. 2 shows a flow diagram showing exemplary steps performed for user verification;

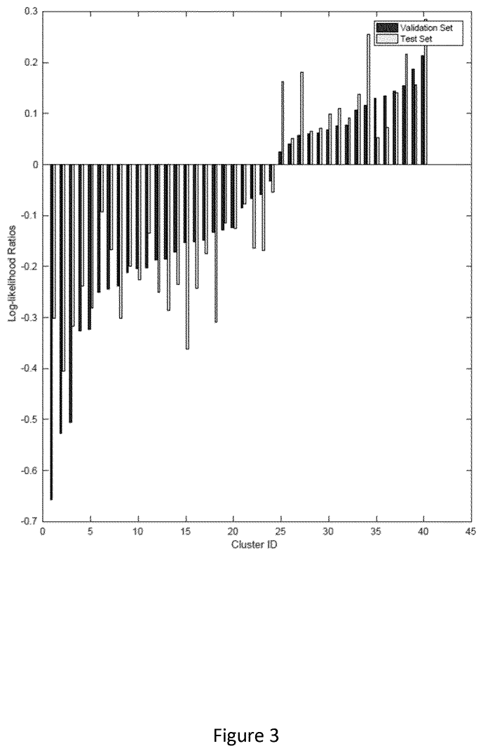

FIG. 3 shows an exemplary diagram of the log-likelihood ratios for a validation set and test set; and

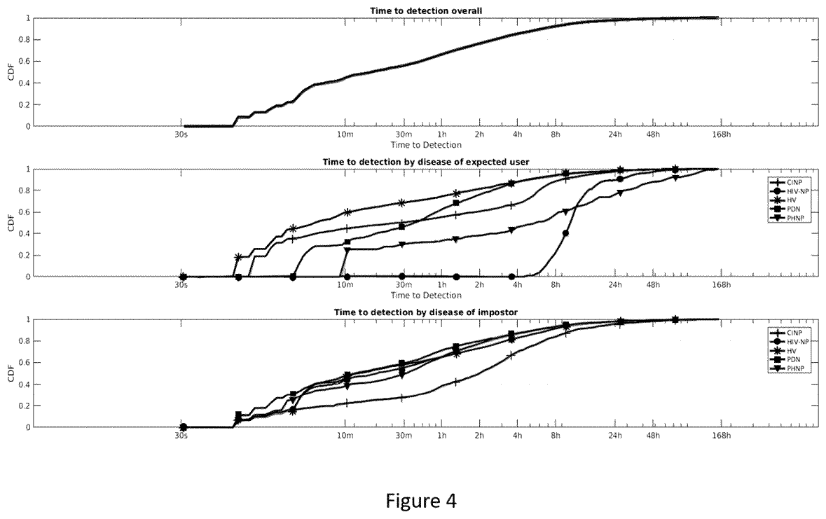

FIG. 4 shows exemplary results of the user verification method in the form of the Time To Detection (TTD) distribution.

FIG. 1 shows an exemplary system 1 comprising a wearable device 2 having a sensor 3, in this example an accelerometer, an optional external device 4 (external to the wearable device), and a data connection 5 connecting the external device and the wearable device, which may particularly be a wireless data connection. The wearable device may be worn on the wrist of a user. However, it is possible that the wearable device is worn on any other body part. As indicated above, the external device is provided optionally. In particular, the wearable device may be configured to perform all the steps of the methods described above. Accordingly, the wearable device may be provided with any hardware and software required for carrying out the method.

The system comprises processing means including a processor 7 comprised in the wearable device and/or a processor 6 comprised in the external device. The processor 7 of the wearable device may be configured to process the data obtained by the accelerometer. In addition or alternatively, the processor 6 comprised in the external device may be configured to process data obtained by the accelerometer and/or data that has already been processed by the processor 7 of the wearable device. The processors each on their own or together may constitute the processing means described above as being configured to carry out the method steps of the methods according to the invention. It should be understood that this is an exemplary system and the invention is not limited to this combination of features.

In the following, a method of creating a user-specific activity model (or fingerprint of activities) for a user and verifying the user by means of the activity model, according to an embodiment of the invention will be described, which may in part or entirely be carried out by a system as shown in FIG. 1 or any other suitable system. In particular, all the method steps may be carried out entirely by the wearable device or all steps except for raising an alarm, which may involve that a signal triggering an alarm is communicated to an external device, which, in response thereto, raises the alarm. It should be noted, as seen in the general part of the description, that the method may also only comprise the creation of the activity model or the verification of the user, that is, the two stages may be performed separately.

The method according to the embodiment may comprise obtaining actigraphy data of a first user, i.e., the expected user, during a first period of time by means of a wearable device. During this time, as described above, the user may optionally be monitored by an additional device or personnel.

The obtained data is a candidate set of raw actigraphy data. This set of raw data is then structured and filtered so as to obtain a data set from which invalid inactivity data has been removed and which provides an undistorted representation of characteristic activities. Thus, a set of good data is obtained, to be used for subsequent steps.

The set of good data is then subjected to a clustering method that groups activities together to form clusters, for example by means of a three-dimensional time series clustering. Thus, activity clusters are provided. The above steps are similarly performed for other users or already available clustered data for other users is retrieved.

Then data from the first user and a subset of data from the other users is used as input for a model generation method. The model generation method provides a probabilistic model that allows for determining, based on observed activities, whether the current user is the expected user or an impostor.

During a second time period after said first time period, actigraphy data of a user currently wearing the wearable device is obtained by means of the wearable device. This may be the expected user or an impostor. During this time, as described above, the user may not, or only infrequently, be monitored by an additional device or personnel.

The actigraphy data obtained during the second time period may then be used to determine, by means of the probabilistic model, the probability that the user wearing the device is the first user. If a first/high threshold is exceeded, it may be determined that the user is in fact the first user. If a second/low threshold is not exceeded, it may be determined that the current user is an impostor. If none of the above is the case, the data may not be characterized as expected user or impostor data until enough data for a characterization is obtained.

When it is determined that actigraphy data is impostor data, it may be marked accordingly and/or an alarm may be triggered.

A flow diagram showing exemplary steps performed for verification is shown in FIG. 2. As can be seen in the flow diagram, each received data segment of user actigraphy data updates the recognition score S. It is determined how typical an observed activity is. If it is typical, S increases, if it is atypical, S decreases, if it is balanced, S remains the same. After updating the recognition score, it is compared to two thresholds. When it is below threshold Tₗ, an alarm is triggered and/or data is marked as impostor data. If it is above the threshold Tₕ, the data belongs to the user. S is reset to 0. Otherwise, more data is collected and the current value of S is updated until one of the criteria is met.

In the following, a method according to the invention will be described in more detail and results obtained using this method are described and visualized by means of FIGS. 3 and 4.

As will be described in more detail below, actigraphy data is grouped into activity clusters by means of a clustering method. This method works on time series with finite length, while the actigraphy data is collected continuously. Hence, prior to clustering activities, the data is organized into consecutive finite time windows, or in other words, the data is subject to a data structuring method.

In this example, adjacent non-overlapping time windows are used. Such windows have the advantage that the results are easier to interpret than for overlapping time windows, as overlaps would consider the same portion of activities multiple times, which generate results that are difficult to interpret. For example, two similar time series with fixed length, which are made of high-frequency cycles, have a high correlation regardless of the time shift. As a consequence, every pair of partially overlapping windows produces high correlation. The opposite happens for signals that are not cyclic: the correlation is high only when the partially overlapping windows include the similar parts. For the time window size, a fixed parameter W is used. A variable time window may alternatively be used in case it is known when the activity starts and ends.

The value of the time series length W may be determined taking into account the consideration that a low value is more likely to trim a unique activity, while a large value could blend different activities together, especially if short-lasting. Experiments showed that a window of 30 seconds (900 samples at 30 Hz) provided good results, although this is not limiting and may vary dependent on various factors.

Furthermore, as will also be discussed below, the clustering method, specifically as its similarity metric is based on Pearson Correlation, is impeded when the standard deviations are on different orders of magnitude. Therefore, a data filtering method is applied that reduces the presence of such data in a manner that does not lead to unacceptable distortions of the overall data set.

In actigraphy data, standard deviations on different orders of magnitude may occur when data is retrieved when the sensors register nearly no movement. There are multiple possible explanations for registering nearly no movement, which include a steadiness period, quiet sleep phase, stuck-at fault, or non-wear.

While the first two may be indicative of the subject's habits and movement characteristics, the second two are not, and produce data that can be characterized as invalid inactivity data.

In the present example, the detection of invalid inactivity data is performed by comparing the standard deviation of the accelerometer magnitude with an activity threshold Tₐ. As seen above, alternatively or in addition to the analysis of the standard deviation, invalid inactivity data may also be identified by means of a non-wear detection algorithm.

The standard deviation allows for determining the degree of variability in the W-long time series; however, if the signal is non-stationary, the variability changes in time. For instance, with a mixture of a constant signal and a small varying signal, the latter will contribute alone for the whole standard deviation. In the present example, this is addressed by a more robust approach, that is, to calculate the standard deviation in M contiguous sub-windows as follows (“robustStd” algorithm):

Algorithm 1: robustStd

| Require: r, Tₐ | |
|---|---|
| Ensure: rubustStd | |
| 1: | W = length(r) |
| 2: | W_{M} = └W/M┘ |
| 3: | stdList = zeros(W_{M}) |
| 4: | for iSubW = 1,...,M do |
| 5: | stdList(iSubW) = std(r((iSubW − 1) W_{M} : iSubW_{M})) |
| 6: | end for |
| 7: | goodStdList = stdList (stdList > Tₐ) |
| 8: | if length(goodStdList) < 0.5 M then |
| 9: | rubustStd = 0 |
| 10: | else |
| 11: | rubustStd = mean(goodStdList) |
| 12: | end if |
| 13: | rubustStd = std(r) |
| 14: | return rubustStd |

Algorithm 1 calculates the standard deviation for each sub-window and checks whether this overcomes the threshold Tₐ. In general, the mean across all the standard deviations that overcome the threshold is returned. However, when less than half of the sub-windows overcome the threshold, the robust standard deviation is set to 0. This is to force the time series to be discarded when it is a mixture of valid and invalid data. For example, each sub-window may be 5 seconds long.

Thus, a criterion to detect low-activity signals is provided, and if the criterion is met, they will not be included in data used for building the model. However, the higher proportion of removed data, the higher distortion in the activity distribution of the selected data, becoming less representative of the real activity distribution. This may result in biased results from the analysis of the accelerometer data and possibly lower accuracy in user verification.

A method for assembling data may be performed so as to produce a dataset of n_{d} days of analysis, whilst minimizing the distortion introduced by the removal of data. To that end, a data assembling criterion may be applied to the data.

The method for assembling data may include replacing missing data with data from other days. The data from other days may be selected by considering similar times of the day, including the same hour of the day.

As an example for producing a dataset of n_{d} days of analysis, for example by means of the method for assembling data, the following steps may be applied. For each hour of data, the data may be added to a good hour dataset if |std(r)>Tₐ|/|std(r)|>Tₐᵣ, wherein Tₐᵣ represents an activity ratio threshold. For each of the 24 hours of the day, it is checked whether there is a respective hour of data in the good hour dataset and, if this is the case, the data is added to the final dataset. Otherwise the hour is marked as an uncharacterized hour, and these steps are repeated for n_{d} times.

This method receives as input the number of days of analysis to select, i.e. n_{d}, and the parameters used to identify a good hour, i.e. Tₐ and Tₐᵣ. Its output is not only the actigraphy dataset, but also the set of uncharacterized hours, i.e. the hours for which there is not enough data. A safe approach to deal with such hours is to discard them for user verification purposes.

Next, activity clustering will be described. Comparing activities quantitatively (e.g., activity intensity distribution) is subject to high variability. Indeed it is normal to have days where the activity is particularly high or low. The invention allows for comparing activities qualitatively, after grouping similar activities together. Time series clustering is used to group portions of actigraphy data that pertain to similar activities.

In general, similar activities can be grouped together through clustering, which identifies similar objects through a similarity/distance metric. In actigraphy, the information contained within an acceleration value is meaningful when contextualized in its previous and future values. For this reason, rather than clustering accelerometer data, accelerometer time series are clustered.

Accelerometer data is particularly suited for shape-based clustering, as the shape provides information about the frequency of the movements, and their temporal concatenation. A clustering technique that focuses on the signals' shape is k-Shape. This is conceptually similar to the more widespread k-Means, but makes use of cross-correlation rather than Euclidean distance to define the distance, or the similarity in this case, between two time series. However, k-Shape is not compatible with the 3-D nature of accelerometer data, and reducing the 3-D information into 1-D, e.g. by calculating the acceleration magnitude, would result in losing the information regarding the direction of acceleration. For this reason, the invention provides a time series clustering method based on the k-Shape method and configured to process three-dimensional accelerometer data.

A second problem of the standard k-Shape method arises from the similarity metric, which is equivalent to calculating the maximum Pearson Correlation, among those obtained by shifting the series in time. This operation is described by the expression below.

$$NCC ( X , Y ) \vert = {MAX}_{δ} \frac{1}{W - 1} {\sum }_{i} \frac{{X}_{i} - \overline{X}}{{s}_{X}} \frac{{Y}_{i + δ} - \overline{Y}}{{s}_{Y}}$$

Where X and Y are the two time series and N is their length. The line above them indicates the sample mean operator, while s is the corrected sample standard deviation.

It should be noted that employing the data filtering described above, which filters out the signals with a particularly low standard deviation while ensuring that the resulting dataset is still representative of the original data, and using k-Shape in cascade to the data filtering technique, overcomes another problem of the Pearson Correlation. That is, in said Pearson Correlation, the normalization factor in the denominator can be particularly low when one of the two signals is nearly constant. This produces noise enhancement, i.e. the normalization is equivalent to increase the noise of the nearly constant signal, say X by a factor s_{Y}/s_{X}. Such noise enhancement can be reduced by the above-described data filtering.

Ae three-dimensional k-Shape-based clustering method, which is suitable for accelerometer data, for example, will be described in the following.

This method allows for an approach that reduces loss of information when compared to clustering data separately on each dimension, which neglects the strong correlation between the three axes, or clustering after reducing the 3-D data to 1-D, e.g. by calculating the magnitude signal, which leads to a loss of information, in particular orientation-related information, e.g., the direction of the acceleration.

The method described herein instead defines the similarity metric as the normalized cross-correlation averaged along the three axes, and maximized among all possible time-shifts, subject to a maximum shift difference across the three axes.

In other words, the similarity metric is an average between three normalized cross-correlations, one for each axis. Across each dimension, the time shifts are selected to maximize the average cross correlation, subject to a constraint in the maximum distance between the three time shifts. Indeed, allowing for different time shifts across the three axes may overcome synchronization errors and allow for more generality in the movements timing, but if the time shifts are significantly different, e.g. more than one second, the activity is different as well.

With respect to the original similarity metric (NCC), the metric configured to process three-dimensional accelerometer data is described below.

$${NCC}^{3 D} ( X , Y ) = {MAX}_{i ∈ \{ 1 , 2 , 3 \} : \vert {δ}_{i} - {δ}_{j} \vert < 1 ∀ i , j} {\sum }_{i = 1}^{3} NCC ( {X}_{i} , {Y}_{i} )$$

Where reference is made to two 3D time series with X and Y. The time series subscript in Xᵢ and Yᵢ identifies the accelerometer data along one of the three dimensions, while (δᵢ is the time shift applied along the i-th dimension. The constraint |δᵢ−δⱼ|<1 requires the time shifts to differ by 1 second at most. The value of 1 second is merely an exemplary constraint. For example, the upper limit of the time shifts may be between 0 and 2 seconds, in particular between 0.2 and 1.8 seconds, in particular between 0.4 and 1.6 seconds, in particular between 0.6 and 1.4 seconds, in particular between 0.8 and 1.2 seconds. These values may in particular be combined with a sampling rate of 30 Hz. In addition or alternatively, the upper limit of the time shifts may be between 0 and 20% of the time window size, in particular between 0 and 10% of the time window size, in particular between 5 and 10% of the time window size. In this case, where, for a given sampling rate, the number of samples obtained within an amount of time that is equal to the upper limit of the time shift (the limit determined as a percentage of the time window size), the value of the allowed time shift will be set to zero.

The above steps allow for clustering actigraphy data into activities with a low level of data loss.

For the purpose of building a model and performing user verification based thereon, suitable data is selected. Among all the activities that are returned by the clustering, the most relevant ones are those where the expected user spends a different time, compared with possible impostors.

In light of the considerations above, it can be concluded that activities with distinctive frequencies are identified more reliably by running the clustering on the data from both the expected user and other persons, who represent the average impostor profile. With data from the expected user only or other persons only, infrequent and frequent activities may be missed (i.e., not assigned to a cluster), respectively.

Thus, after performing the data structuring and filtering steps described above, the clustering is performed on data from the expected user and on data of other persons. When Nₑᵤ samples are available from the expected user, Nₑᵤ/Mᵢᵤ samples will be selected for the other persons, where Mᵢᵤ is the number of persons that are used to build the reference profile.

For the expected user, the Nₑᵤ samples are selected from the first period, also referred to as reference period. For instance, in a clinical trial, the first period may correspond to the baseline period. The samples for the reference profile can be taken from different participants of a study, which may or may not be the same study to which the expected user belongs. For each of them, Nₑᵤ samples are initially collected, and thereafter Nₑᵤ/Mᵢᵤ are samples drawn at random, for example with uniform probability.

It is possible to merge data from the expected user and other persons and then performing the clustering. Alternatively, the clustering may be performed separately on actigraphy data from the expected user and Mᵢᵤ other persons and the results of the clustering may then be merged.

The method may comprise creating, fitting, and refining a probabilistic model or it may use a pre-existing probabilistic model for user verification. An example for creating the probabilistic model, and fitting and refinements thereof are described in the following.

With the clustering procedure described above, a set of centroids is obtained, which is used to group similar activities together. The time spent by the user in each cluster is connected to the frequency of each activity, which in turn is used to verify the user of the actigraphy device. Indeed, the activity frequencies are connected to the person's physical fitness, lifestyle, routine, and to the patterns in performing each activity, which are all possibly distinctive characteristics.

Thus, it is estimated how much information is given by the frequency of each activity and this estimation is used it to verify the user of the actigraphy device.

The probabilistic model is built to that end, where the goal is to characterize a random variable U, which represents the user of the actigraphy device, and can assume the values “expected user” or “impostor”. The value of U will change over time if there are changes of user. Therefore, its evolution in time is characterized. In particular, it is characterized as a function of the observed activities (clusters), therefore with a time granularity of W.

If the user quickly hands over the sensors to an impostor, or vice versa, the accelerometer data within the respective time window may be a mixture of user and impostor data.

The time-ordered sequence of traversed clusters is modelled with the random process {C₁, C₂, . . . , Cₜ}, where the value of each realization Cᵢ is one among the k clusters c₁, . . . , cₖ, given in output by the clustering method.

The confidence is defined as the probability of expected user given the history of observed clusters until the current time instant t. The confidence corresponds to: Pᵣ (U=uₑₓₚ|C₁, C₂, . . . , Cₜ). The expected user is indicated with uₑₓₚ. Data is classified as impostor data when Pᵣ (U=uₑₓₚ|C₁, C₂, . . . , Cₜ)<Tₗ, where Tₗ is a low threshold, i.e. it tests when the confidence is low enough to infer the presence of an impostor. The value of Tₗ may be automatically adjusted to optimize a performance metric.

The distribution of the confidence is characterized as a function of the cluster history as follows.

First, the scenario of observing only one cluster is addressed. For convenience of notation, the following alias is introduced: θⱼ=Pr(U=Uₑₓₚ|C₁=cⱼ).

For a generic cluster j, the random variable (U|C₁=cⱼ) follows a Bernoulli distribution, characterized by the only parameter θⱼ. The value of such a parameter can be estimated from the cluster occurrences. N time series extracted from the data of the expected user data and as many time series extracted from the data of the impostors are considered. N_{j,eu} and N_{j,iu}, which correspond to the occurrences of cluster c, for the expected user and the impostors, respectively are calculated next. If θⱼ were known, the likelihood of observing such cluster occurrences would be: Pr(N_{j,eu},N_{j,iu}|θⱼ)=(θⱼ)^{Nj,eu}(1−θⱼ)^{Nj,iu}.

At this point, θⱼ could be estimated by maximizing the quantity above. This approach (known as maximum-likelihood estimation), would lead to

$${\hat{θ}}_{j}^{ML} = \frac{{N}_{j , {e}_{u}}}{{N}_{j , {e}_{u}} + {N}_{j , {i}_{u}}}$$

This approach may have problems related to rare clusters, i.e. clusters that are not rich with samples. For instance, if a cluster is never observed for the expected user, a zero probability would be obtained even if the same cluster is observed only once for the impostor. A more robust approach consists of defining a prior probability distribution for θⱼ and maximizing the posterior, i.e. the product of likelihood and prior. The result of this approach, known as the Maximum A-Posteriori (MAP) criterion, follows more the observations when there are more samples, and is closer to the prior with fewer data samples.

When the likelihood is Bernoulli-distributed, it is common to assume a Beta distribution for the prior, because the product of a Bernoulli and a Beta distribution is, again, a Beta distribution (which is said to be the conjugate prior of the Bernoulli distribution).

The Beta distribution is characterized by two parameters: α and β. These parameters determine essentially two effects: the intensity of the prior effect, and the bias towards 0 or 1 of θⱼ.

In the present example, it is assumed that users and possible impostors are equally likely. This corresponds to having α=β, which implies that the expected value of the probability θⱼ, prior to any observation, is 0.5.

As a result, the expected value of the Beta posterior probability, calculated with the MAP criterion, is equal to:

$${\hat{θ}}_{j} = \frac{{N}_{j , {e}_{u}} + α - 1}{{N}_{j , {e}_{u}} + {N}_{j , {i}_{u}} + 2 α - 2} \qquad ( 1 )$$

The role of α is particularly evident when a cluster is unobserved for the user. In this case, the cluster assumes a non-zero probability equal to

$$\frac{α - 1}{{N}_{{e}_{j} , I} + 2 α - 2} • .$$

Note that the impact from the prior decreases with the number of samples. Conversely the prior has more effect as a increases (for example, α=30 may be used).

Having concluded the analysis with one observed cluster, the scenario with two consecutive clusters, C₁ and C₂ is analyzed next. The confidence after observing the second cluster is:

$$Pr ( U = {u}_{exp} \vert {C}_{1} , {C}_{2} ) = \frac{Pr ( {C}_{2} \vert {C}_{1} , U = {u}_{exp} ) Pr ( U = {u}_{exp} \vert {C}_{1} )}{Pr ( {C}_{2} \vert {C}_{1} )}$$

The value taken by C₁ and C₂ has been omitted for convenience of notation. In this equation, the probability Pᵣ (U=uₑₓₚ|C₁) coincides with the probability estimated for the scenario with one observed cluster, i.e., it is the confidence at the previous step. The remaining terms, instead are the ratios between two likelihoods, one calculated with the knowledge that the monitored subject is the user, and the other without such knowledge. The above expression holds in general, indeed:

$$Pr ( U = {u}_{exp} \vert {C}_{1} , … , {C}_{t} ) = \frac{Pr ( {C}_{t} \vert {C}_{1} … {C}_{t - 1} , U = {u}_{exp} ) Pr ( U = {u}_{exp} \vert {C}_{1} , … , {C}_{t - 1} )}{Pr ( {C}_{t} \vert {C}_{1} … {C}_{t - 1} )}$$

Here, the right term in the numerator is, again, the confidence calculated at the previous step. The other two terms need to be estimated from the data, with an adequate number of observations. However, this is difficult when activities are performed with low frequency, e.g. brushing teeth, an activity which is generally performed two or three times per day. Assuming that such an activity corresponds to cluster c_{b} even estimating the likelihoods with memory equal to 1, i.e. all the Pr(Cᵢ=c_{b}|Cᵢ₋₁=cₐ), may be difficult. The estimate is more reliable when the data covers a long time frame with respect to the number of clusters. With a number of clusters in the order of 50 and a data time frame of one week, for example, it would be preferable to estimate each likelihood Pr(Cᵢ|Cᵢ₋₁) on one sample, on average.

For the reasons described above, Pr(Cᵢ|C₁, . . . , Cᵢ₋₁) is approximated with Pr(Cᵢ), and Pr(Cᵢ|C₁, . . . , Cᵢ₋₁, U=uₑₓₚ) with Pr(Cᵢ|U=uₑₓₚ).

Thus, the expression

$$Pr ( U = {u}_{exp} \vert {C}_{1} , … , {C}_{t} ) \approx \frac{Pr ( {C}_{i} \vert U = {u}_{exp} ) Pr ( U = {u}_{exp} \vert {C}_{1} , … , {C}_{i - 1} )}{Pr ( {C}_{i} )} \qquad ( 2 )$$

gives a memory-less model with respect to the cluster time series. However, the confidence still has memory.

The quantities Pr(Cᵢ|U=uₑₓₚ)/Pr(Cᵢ) are referred to as likelihood ratios.

These are calculated through the application of Bayes theorem.

$$Pr ( U = {u}_{exp} \vert {C}_{i} ) = \frac{Pr ( {C}_{i} \vert U = {u}_{exp} ) Pr ( U = {u}_{exp} )}{Pr ( {C}_{i} )} ⇒ \frac{Pr ( {C}_{i} \vert U = \vert {u}_{exp} )}{Pr ( {C}_{i} )} = \frac{Pr ( U = {u}_{exp} \vert {C}_{i} )}{Pr ( U = {u}_{exp} )}$$

With digital computations, it is more convenient to work with logarithms, which transform the products into sums and reduce the risk of overflow/underflow. The resulting log-likelihood ratios are the only quantities that need to be stored for the model, and are calculated as

llr(cⱼ)=log(Pr(U=uₑₓₚ|Cᵢ=cⱼ))−log(Pr(U=uₑₓₚ)) (3),

where llr stands for log-likelihood ratio. The term to the left corresponds to θ, which is fitted to the data with (1), whereas the term to the right is the a-priori confidence with no observation.

Since Pr(U=uₑₓₚ|Cᵢ=cⱼ) was assumed to have an expected value of 0.5, also the expected value of Pr(U=uₑₓₚ), which is the weighted average across all clusters, is equal to 0.5.

The probabilistic model may be fitted by counting the number of occurrences for the expected user and for the impostor in each cluster. However, the impostor data is unknown and, as such, it is substituted with data collected from other persons. The data used for fitting the probabilistic model, is extracted from the initial days (e.g. baseline of a clinical trial) of analysis of the expected users (first period) and the other persons as well, as explained above. As a consequence, it can be assumed that, during this period, the user of the actigraphy device is the expected one.

Once the probabilistic model is fitted, the log-likelihood ratios are saved in memory and used to verify the user. The model goodness in the user verification task depends on how representative the model is for the data that is yet to be observed. To measure this, the log-likelihood ratios can be calculated onto two datasets collected at different and non-overlapping times. In FIG. 3, the log-likelihood ratios calculated for a real user of a study during two different observational periods are shown, denoted with validation set and test set. In FIG. 3, positive and negative bars represent clusters that increase and decrease the confidence, respectively. Clusters where the consistency criterion is not satisfied have been expunged from this graph, as their log-likelihood is zero.

The comparison between validation and test set allows for evaluating the stability of the log-likelihoods, i.e. the consistency in time of the time spent in each cluster. The closer the two values for each cluster, the higher the predictability of the user in the performed activities.

In terms of user verification accuracy, preferably the log-likelihoods are not only stable, but also significantly different from zero. The log-likelihood ratios are used each time a cluster is observed, to update the confidence in the user's identity. The confidence update criterion is obtained by combining (3) and (2), which give the following expression:

log(Pr(U=uₑₓₚ|C₁, . . . ,Cᵢ))≈log(Pr(U=uₑₓₚ|C₁, . . . ,Cᵢ₋₁))+llr(Cᵢ).

The model may further be refined as described in the following. The problems with a memory-less model may arise when activities are performed with considerably different frequencies, i.e. differing by one order of magnitude or more. For instance, assume that the user traverses cluster c₁ twenty times per day, whereas the average person traverses it two hundred times per day. If, on one day, both clusters have been traversed twenty times, the probability that the next cluster is c₁ is much less than the probability that it is c₂, unless there is an impostor. Nevertheless, the memory-less model abstracts from this information, hence the probabilities stay the same, regardless of the observations.

A drawback of this effect is that the observations of cluster c₂ count ten times as much as the observations of cluster c₁. To counteract this effect, the probabilities are adjusted through their average number of occurrences. To do that, the llr values are divided by the average number of occurrences for that cluster, i.e., llr^{norm}(cⱼ)=llr(cⱼ)/N_{cj,P}.

In the previous example, assuming that c₁ and c₂ have the same llr values, the value of llr^{norm}(c₂) will be ten times smaller than the value of llr^{norm}(c₁). As a consequence, two hundred observations of c₂ will impact as much as twenty observations of c₁.

Another problem may arise when the frequencies of each cluster are highly inconsistent. For instance, the expected user may perform an activity during weekends only, whereas the average impostor would take them with equal frequency throughout the week. In this case, it is likely that the llr is negative. However, during weekends, a high frequency of that activity will be registered for the user, which leads to a confidence decrease.

To take these effects into account, llr may be calculated on a daily basis and it may be checked that the values have the same sign for at least 75% of days during the reference period. If that is not the case, that cluster is excluded to prevent it from affecting the confidence. Further improvements may be obtained in this regard by building an alternative model for different days, such as non-working days.

The verification step may be performed as follows.

After having organized the accelerometer data into time-limited signals, clustering them to identify similar activities, and calculating the probabilistic impact of observing each activity, which is used to infer whether the observed user is the expected one, has been performed, the steps for verifying the expected user can be performed, e.g., during the second time period. To perform the actual user verification step, an inference is made about the user's identity.

For example, in the verification method, when the probability is high enough the data may be marked as correct and when it is low enough the data is marked as impostor data. To do so, the probability is compared to a low threshold Tₗ and a high threshold Tₕ. These thresholds may be used as decision criteria.

In the following, it is shown how such decision criteria are applied in this example based on the information extracted from the raw accelerometer data, i.e., assigning it to the closest cluster, updating the current user log-probability by adding the log-likelihood ratio corresponding to that cluster, and inferring the user's identity when there is enough confidence.

Algorithm 2: ImpostorDetection

Require: xyz, C, W, IIr^{norm}, maxE, Tₗ,Th
Ensure: I

| | |
|---|---|
| 1: | SET I to EMPTY ARRAY with SIZE length(xyz) {Initialise Output} |
| 2: | iSample = 1 |
| 3: | SET sampleWindow to EMPTY LIST |
| 4: | evidenceSize = 0 |
| 5: | evidenceStart = 1 |
| 6: | PrP = log(0.5) {Initialise current probability with the prior} |
| 7: | while iSample <= length(xyz) do |
| 8: | APPEND xyz(iSample) to sampleWindow |
| 9: | if length(sampleWindow) = W then |
| 10 | Cᵢ = argmax(NCC^{3D}(C,sampleWindow)) |
| 11 | SET sampleWindow to EMPTY LIST |
| 12 | PrP = PrP + Ilr^{norm}(Cᵢ) |
| 13 | evidenceSize = evidenceSize + 1 |
| 14 | classified = false |
| 15 | if PrP<Tₗ then |
| 16 | l(evidenceStart : iSample) = 1 |
| 17 | classified = true |
| 18 | else if PrP>Tₕ OR evidenceSize > maxE then |
| 19 | l(evidenceStart : iSample) = 0 |
| 20 | classified = true |
| 21 | end if |
| 22 | if classified then |
| 23 | evidenceSize = 0 |
| 24 | evidenceStart = iSample+1 |
| 25 | PrP = log(0.5) |
| 26 | end if |
| 27 | end if |
| 28 | iSample = iSample + 1 |
| 29 | end while |
| 30 | return I |

The value returned by this method in this example is the array I, which contains a value 1 in correspondence of an impostor sample, and a value 0 otherwise. The user verification check is applied each time W samples are observed, where W corresponds to the size of the length of the cluster centroids. The closest centroid, calculated with the similarity metric described above, determines the activity of the current window. A log-likelihood ratio is associated with the recognized activity, and it is added to the current confidence. A classification is made if the updated confidence is lower than Tₗ or higher than Tₕ. If neither is the case, more evidence is collected; however, after maxE confidence updates, the evidence is reset. Indeed, evidence that is particularly old may become less significant, as the user may have changed in the meantime. The parameters maxE, Tₗ, and Tₕ are the ones that drive the performance of the user verification algorithm 2, and may be optimized with respect to a performance metric like the rate of raising false alarms or rate of not raising true alarms or time to detection of an impostor.

An example for determining the performance of the verification method and experimental results is described in the following.

The performance of a user verification technique is a combination of the gain in detecting impostors and the cost of detecting genuine users as impostors. There exists a tradeoff between the two where the tuning parameters may be one or more of the above identified parameters maxE, Tₗ, and Tₕ.

For the following considerations, it is assumed that the main objective is to raise an alarm in the face of malicious data, as well as identifying the measurements where the impostor was wearing the sensors. Alarms are useful to, e.g., ask the users to take the device back, while identifying impostor data can be used to cleanse the data.

The performance metric used in the following is a combination of the cost of raising false alarms, and of the cost of not raising true alarms, or raising them in delay.

The False Alarm, Rate (FAR) is defined as the expected number of false alarms that occur in a period of time. As an example, in practice, FAR should be in the order of one per year. Indeed, false alarms may cause genuine data to be discarded, or initiate a manual check of the relevant data, which is time-consuming.

The Mean Time To Detection (MTTD) is defined as the expected delay between the time when the user hands the device over to the impostor, and the time when this is detected. In the following, while keeping the FAR below the above value, the MTTD metric was minimized.

Experimental results for one exemplary embodiment are explained in the following. The experiments were conducted with accelerometer data from non-interventional studies where the participants (users) were wearing the Actigraph GT9X/Link device on their wrist (they were allowed to choose between left or right), which was configured to capture accelerometer data at 30 Hz, whilst being worn on the wrist throughout the study period. Raw acceleration data was extracted at the end of the studies through the USB interface.

For each of the participants, the clustering is run on the data collected during a reference period (first period), belonging both to that participant and to other participants (in a proportion of 1 to 1). The clustered data from the other participants is used to build a reference profile of possible impostors, and is selected among the participants of different studies. This is to reduce bias connected to the recruitment criteria of the specific study taken by the correct participant. For instance, if the expected user belongs to a study of patients with Osteoarthritis, using data from the same study to run the clustering would be detrimental to detecting patterns that are specific to other diseases.

The clustering was run on a balanced mixture of expected user data and other persons' data, which was collected during different studies. Then, the probabilistic model was built as explained above.

In order to test the verification method (particularly the Algorithm 2 described above), the FAR and MTTD are calculated for each user. The FAR is calculated by running Algorithm 2 on the user's data, excluding the portion pertaining to the reference period (first period).

To calculate the MTTD, the presence of impostor data is simulated by using the data from the other participants of the same study. As conveyed above, participants from the same study are likely to share common characteristics, however if an impostor shares similar characteristics with the user, the detection task is more difficult, but still reliable. This works as a conservative measure, i.e. some kind of lower bound to the actual performance.

Impostor data could be simulated by stitching two pieces of accelerometer data, one from the correct user, and the other from a different subject. However, it is safe to abstract from the transition period, which may be in the order of 30 seconds, i.e. only one piece of evidence. During this transitory period, the data will be assigned to an unpredictable cluster, as the activity of “giving the sensors to someone else” was probably never observed during clustering. Thus, it can be assumed that its effect will be minimal and the impostor data can be processed as if the data collection started when the impostor took the sensors. As soon as the impostor is detected, the time it took is calculated and a TTD sample is recorded. Then, the same procedure is repeated with the following data, the confidence is reset, and, when the detection triggers add a new TTD sample. At the end of this process an average among all TTDs is determined to extract the MTTD metric.

When setting the FAR to zero as a constraint, and minimizing the MTTD under this constraint, a distribution of the TTD averaged across all possible pairs of users and impostors as shown in FIG. 4 is obtained. The distribution plot is an empirical Cumulative Distribution Function, hence the correct way to interpret it is that the fraction of impostors on the y-axis can be detected within a time less than the respective value on the x-axis.

As can be seen from the Figure, about 35% impostors are detected in less than 30 minutes, about 77% are detected in less than 1 hour, about 90% are detected in less than 2 hours, and all of them are detected in less than 8 hours.

Although the previously discussed embodiments and examples of the present invention have been described separately, it is to be understood that some or all of the above-described features can also be combined in different ways. The above-discussed embodiments are not intended as limitations, but serve as examples, illustrating features and advantages of the invention.

1. A computer-implemented method for providing a user-specific activity model, the method comprising:
obtaining actigraphy data of a plurality of users and
determining the user-specific activity model of a first user of the plurality of users based on the actigraphy data of the first user and on a reference actigraphy data set comprising actigraphy data of the remaining users of the plurality of users.

2. A computer-implemented method for user verification, comprising:
obtaining actigraphy data by means of a wearable device,
verifying, based on a user-specific activity model of a first user, which is based on actigraphy data of the first user obtained during a first period of time, whether actigraphy data obtained during a second period of time subsequent to the first period of time belongs to the first user, and
in response to a determination that any of the actigraphy data obtained during the second period of time does not belong to the first user, marking the data that does not belong to the first user as impostor data and/or raising an alarm indicating that impostor data was detected.

3. The method according to claim 2, further comprising:
determining the user-specific activity model of the first user based on the actigraphy data of the first user and on a reference actigraphy data set comprising actigraphy data of a plurality of users.

4. The method according to claim 3, wherein actigraphy data obtained by the wearable device is added to a candidate set of actigraphy data and the method further comprises structuring and/or filtering the actigraphy data of the candidate set to obtain a data set to be used for creating the activity model and/or for the verifying step.

5. The method according to claim 4, wherein the structuring comprises dividing the actigraphy data of the candidate set into consecutive finite time windows.

6. The method according to claim 4, wherein the filtering comprises a step of removing data that is categorized as invalid inactivity data from the candidate set.

7. The method according to claim 4, wherein the filtering comprises characterizing a sub-set of data within the candidate set as good data only when the proportion of data removed from the sub-set exceeds a threshold Tₐᵣ and/or only when the sub-set is part of a group of similar sub-sets occurring repeatedly in a specific pattern, and adding only the good data to a final data set, wherein the final data set is used.

8. The method according to claim 7, further comprising processing actigraphy data from the final data set, so as to group activities together to form clusters by means of a three-dimensional time series clustering method, to provide activity clusters.

9. The method according to claim 2, wherein the verifying comprises:
inputting actigraphy data obtained during the second period of time into a probabilistic model that defines the probability that the user wearing the device is the first user based on the user-specific activity model and an activity in the actigraphy data,
determining whether the probability determined by the probabilistic model is above a first threshold, also referred to as high threshold Tₕ and/or determining whether the probability determined by the probabilistic model is below a second threshold, also referred to as low threshold Tₗ,
determining that the actigraphy data belongs to the first user when the probability determined by the probabilistic model is above the first threshold, and/or determining that the actigraphy data does not belong to the first user when the probability determined by the probabilistic model is below the second threshold, and/or determining that the input data is not sufficient for determining whether the actigraphy data belongs to the first user or an impostor when the first threshold is not exceeded and the second threshold is exceeded.

10. The method according to claim 9, wherein the probabilistic model is configured to update the probability based on each observed activity, and wherein the verifying comprising repeatedly determining whether the probability determined by the probabilistic model is above a first threshold and/or determining whether the probability determined by the probabilistic model is below a second threshold until the probability either exceeds the first threshold Tₕ or does not exceed the second threshold Tₗ.

11. The method according to claim 3, further comprising determining a plurality of preliminary activity models from the actigraphy data obtained during the first time period and employing the plurality of preliminary activity models to obtain the user-specific activity model by generating a consensus activity model from the preliminary activity models or by employing the plurality of preliminary activity models to remove a portion of the actigraphy data obtained during the first period of time, for example actigraphy data identified as likely impostor data, to obtain a reduced set of actigraphy data, which is used for obtaining the user-specific activity model.

12. A system for providing a user-specific activity model comprising processing means configured to perform the following steps:
obtaining actigraphy data of a plurality of users, and
determining the user-specific activity model of a first user of the plurality of users based on the actigraphy data of the first user and a reference actigraphy data set comprising actigraphy data of the remaining users of the plurality of users.

13. A system for user verification, comprising:
a wearable device, comprising:
a sensor configured to obtain actigraphy data, and
one or more processors, and
memory storing the one or more programs configured to be executed by the one or more processors, the one or more programs including instructions for:
verifying, based on a user-specific activity model of a first user, which is based on actigraphy data of the first user obtained during a first period of time, whether actigraphy data obtained during a second period of time subsequent to the first period of time belongs to the first user, and
in response to a determination that any of the actigraphy data obtained during the second period of time does not belong to the first user, marking the data that does not belong to the first user as impostor data and/or raising an alarm indicating that impostor data was detected.

14. The system according to claim 13, wherein the one or more programs include further instructions for:
determining the user-specific activity model of the first user based on the actigraphy data of the first user and on a reference actigraphy data set comprising actigraphy data of a plurality of users.

15. The system according to claim 13, wherein actigraphy data obtained by the wearable device is added to a candidate set of actigraphy data and the method further comprises structuring and/or filtering the actigraphy data of the candidate set to obtain a data set to be used for creating the activity model and/or for the verifying step.

16. The system according to claim 15, wherein the structuring comprises dividing the actigraphy data of the candidate set into consecutive finite non-overlapping time windows.

17. The method according to claim 15, wherein the filtering comprises a step of removing all data that is categorized as invalid inactivity data from the candidate set.

18. The system according to claim 15, wherein the filtering comprises a step of removing all data that is categorized as invalid inactivity data from the candidate set.

19. The system according to claim 15, wherein the filtering comprises characterizing a sub-set of data within the candidate set as good data only when the proportion of data removed from the sub-set exceeds a threshold Tₐᵣ and/or only when the sub-set is part of a group of similar sub-sets occurring repeatedly in a specific pattern, and adding only the good data to a final data set, wherein the final data set is used for creating the activity model.

20. The system according to claim 19, wherein the one or more programs including instructions for processing actigraphy data from the final data set, so as to group activities together to form clusters by means of a three-dimensional time series clustering method based on a k-partition method, to provide activity clusters.
